SEQUENCE LISTING (17 sequences)

SEQ ID NO: 1 - protein, 222 aa, Artificial Sequence
  Description of Artificial Sequence Synthetic polypeptide
MKIKLRMEGDVNGHPFVITGEGSGKPYEGTQTVDLKVKEGGPLPFAYDILTVAFQYGNRAFTKYPADIPDYFKQSFPDGYCWERSMVFEDQGSCVVKSVISLDKKEPDCFIYDIRFKGKNFPATGPIMQKETVKWDASTQRMYERDGVLVGDAKMKLKLKGGGHYRVDIKSTYRAKGVVQYMPGNHYVDHHIEILHHDKDYNSVTVYESAEARHCRPSSKAE

SEQ ID NO: 2 - protein, 13 aa, Artificial Sequence
  Description of Artificial Sequence Synthetic peptide
  MOD_RES (1)..(1): Lys or Arg
XKISASEFDRPLR

SEQ ID NO: 3 - protein, 5 aa, Artificial Sequence
  Description of Artificial Sequence Synthetic peptide
DRPLR

SEQ ID NO: 4 - protein, 9 aa, Unknown
  Description of Unknown MARCKS phosphorylation recognition motif sequence
KKKKRFSFK

SEQ ID NO: 5 - protein, 9 aa, Unknown
  Description of Unknown synapsin phosphorylation recognition motif sequence
LRRLSDSNF

SEQ ID NO: 6 - protein, 10 aa, Unknown
  Description of Unknown glycogen synthase phosphorylation recognition motif sequence
KKLNRTLTVA

SEQ ID NO: 7 - protein, 10 aa, Artificial Sequence
  Description of Artificial Sequence Synthetic peptide
KKANRTLSVA

SEQ ID NO: 8 - protein, 10 aa, Artificial Sequence
  Description of Artificial Sequence Synthetic peptide
  MOD_RES (6)..(9): Any amino acid
LAQRRXXXXL

SEQ ID NO: 9 - protein, 5 aa, Artificial Sequence
  Description of Artificial Sequence Synthetic peptide
  MOD_RES (3)..(3): Any amino acid
LAXRR

SEQ ID NO: 10 - protein, 8 aa, Artificial Sequence
  Description of Artificial Sequence Synthetic peptide
SDASSCES

SEQ ID NO: 11 - protein, 5 aa, Artificial Sequence
  Description of Artificial Sequence Synthetic peptide
RRLFG

SEQ ID NO: 12 - protein, 6 aa, Artificial Sequence
  Description of Artificial Sequence Synthetic 6xHis tag
HHHHHH

SEQ ID NO: 13 - protein, 12 aa, Artificial Sequence
  Description of Artificial Sequence Synthetic peptide
EEEIYGEFEAKK

SEQ ID NO: 14 - protein, 5 aa, Artificial Sequence
  Description of Artificial Sequence Synthetic peptide
  MOD_RES (4)..(4): Any amino acid
IYAXP

SEQ ID NO: 15 - protein, 9 aa, Artificial Sequence
  Description of Artificial Sequence Synthetic peptide
AEVIYAAPE

SEQ ID NO: 16 - protein, 9 aa, Artificial Sequence
  Description of Artificial Sequence Synthetic peptide
PPYPPPPIP

SEQ ID NO: 17 - protein, 12 aa, Artificial Sequence
  Description of Artificial Sequence Synthetic peptide
RKRERAYSFGHH

(19) United States
(12) Patent Application Publication
(10) Pub. No.: US 2023/0064490 A1
(43) Pub. Date: Mar. 2, 2023

(54) FLUORESCENT PROTEIN BIOSENSOR FOR DETECTION OF KINASE ACTIVITY

(71) Applicant: Lucidicor Inc., Providence, RI (US)

(72) Inventor: Hung-Teh Kao, Providence, RI (US)

(21) Appl. No.: 16/923,927

(22) Filed: Jul. 8, 2020

Related U.S. Application Data
(63) Continuation of application No. PCT/US2019/012645, filed on Jan. 8, 2019.
(60) Provisional application No. 62/614,678, filed on Jan. 8, 2018.

(51) Int. Cl. G01N 33/533; C07K 14/435; C12Q 1/42; C12Q 1/48; C12N 9/12

(52) U.S. Cl. CPC G01N 33/533; C07K 14/43595; C12Q 1/42; C12Q 1/485; C12N 9/12; C07K 2319/09; C07K 2319/60; G01N 2500/04; G01N 2800/52; G01N 2800/54; G01N 33/574

(57) ABSTRACT

Provided by the present disclosure are fluorescent proteins that can detect kinase activity. The fluorescent proteins of the present disclosure have us in, for example, detecting kinase activity in any eukaryotic cell.

### RELATED APPLICATIONS

This application claims priority to U.S. Provisional Application No. 62/614,678, filed on Jan. 8, 2018, the entire contents of which is incorporated herein by reference in its entirety.

### BACKGROUND OF THE INVENTION

Protein kinases are a class of important enzymes that catalyze the transfer of a phosphate group from ATP to serine, threonine, tyrosine, and histidine residues of peptides and proteins. The specificity of kinases is controlled by kinase recognition motifs, which are amino acid residues surrounding the amino acid to be phosphorylated. The ubiquitous process of protein phosphorylation is central to signal transduction and regulation in living organisms. By catalyzing transfer of the γ-phosphoryl group of ATP to the side chains of serine, threonine, and/or tyrosine, protein kinases play an important role in regulating many aspects of cellular function in eukaryotes, including proliferation, cell cycle, metabolism, transcription, and apoptosis. Not surprisingly, protein kinases have also emerged as attractive targets for drug discovery, since many are associated with a wide variety of diseases, from cancer to inflammation. Thus, tools that allow for monitoring of kinase activity are in great demand in both pharmaceutical and academic settings.

The present disclosure provides novel fluorescent biosensors for detection of kinase activity.

### SUMMARY OF THE INVENTION

The present disclosure describes an engineered fluorescent protein (PhosFluor) that displays increased brightness in response to phosphorylation.

According to a first aspect, the present disclosure features an isolated fluorescent polypeptide (PhosFluor) comprising an amino acid sequence at least 90% identical to SEQ ID NO: 1 (MKIKLRMEGDVNGHPFVITGEGSGKPYEGTQTVDLKVKEGGPLPFAY DILTVAFQYGNRAFTKYPADIPDYFKQSFPDGYCWERSMVFEDQGSCVVKSVISLDK KEPDCFIYDIRFKGKNFPATGPIMQKETVKWDASTQRMYERDGVLVGDAKMKLKLK GGGHYRVDIKSTYRAKGVVQYMPGNHYVDHHIEILHHDKDYNSVTVYESAEARHC RPSSKAE), and variants thereof. According to one embodiment, the amino acid sequence is at least 95% identical to SEQ ID NO: 1. According to one embodiment, the amino acid sequence is at least 96% identical to SEQ ID NO: 1. According to one embodiment, the amino acid sequence is at least 97% identical to SEQ ID NO: 1. According to one embodiment, the amino acid sequence is at least 98% identical to SEQ ID NO: 1. According to one embodiment, the amino acid sequence is at least 99% identical to SEQ ID NO: 1. According to one embodiment, the amino acid sequence corresponds to SEQ ID NO: 1. According to one embodiment, SEQ ID NO:1 comprises one or more mutations. According to another embodiment, the mutation comprises the addition of a substrate recognition site. According to one embodiment, the protein further comprises at least one kinase recognition sequence. According to another embodiment, the kinase recognition sequence is fused to the N-terminus of SEQ ID NO:1. According to another embodiment, the kinase recognition sequences recognizes protein kinase A (PKA). According to a further embodiment, the kinase recognition sequence recognizes a kinase selected from the group consisting of Protein Kinase A, Protein Kinase C, Ca2+/calmodulin-dependent protein kinase I, Ca2+/calmodulin-dependent protein kinase II, and Abl. According to one embodiment, the protein further comprises at least one protein kinase docking site. According to one embodiment, the protein further comprises at least one targeting sequence that directs the protein to a specific cellular compartment. According to one embodiment of the above aspects and embodiments, the isolated fluorescent polypeptide further comprises a second fluorescent molecule. According to one embodiment, the second fluorescent molecule is a fluorescent protein or a quantum dot.

According to another aspect, the present disclosure features an isolated fluorescent polypeptide protein comprising an amino acid sequence at least 90% identical to SEQ ID NO: 1 and at least one kinase recognition sequence. According to one embodiment, the kinase recognition sequence is fused to the N-terminus of SEQ ID NO:1. According to one embodiment, the kinase recognition sequences recognizes protein kinase A (PKA).

According to another aspect, the present disclosure features an isolated fluorescent polypeptide comprising an amino acid sequence at least 90% identical to SEQ ID NO: 1 and at least one protein kinase docking site.

According to another aspect, the present disclosure features an isolated fluorescent polypeptide comprising an amino acid sequence at least 90% identical to SEQ ID NO: 1 and at least one targeting sequence that directs the protein to a specific cellular compartment.

According to one embodiment, the isolated fluorescent polypeptide of any one of the above aspect further comprises a second fluorescent molecule. According to one embodiment, the second fluorescent molecule is a fluorescent protein or a quantum dot. According to one embodiment, the present disclosure features a nucleic acid encoding the isolated fluorescent polypeptide of any one of the above aspects or embodiments. According to one embodiment, the present disclosure features a cell comprising the nucleic acid.

According to another aspect, the present disclosure features a method for determining whether a sample contains an activity, comprising obtaining a sample; contacting the sample with the fluorescent polypeptide of any one of the aspects and embodiment herein; and measuring the amount of fluorescence from said fluorescent polypeptide. According to one embodiment, the activity is kinase activity. According to one embodiment, the activity is phosphatase activity. According to another embodiment, the method further comprises the step of comparing the amount of fluorescence measured from said fluorescent polypeptide with the amount of fluorescence from a control sample. According to one embodiment, the sample is selected from the group consisting of: a blood sample, a tissue sample or a tumor biopsy. According to one embodiment, the sample is from a subject suffering from a disease or disorder. According to another embodiment, the disease or disorder is cancer. According to one embodiment, measuring the amount of fluorescence is used to determine changes in subcellular localization, relative abundance or activation kinetics. According to one embodiment, measuring the amount of fluorescence is used to determine risk of metastasis or resistance to therapeutic agents.

According to another aspect, the present disclosure features a method of screening for a compound that has kinase activity, comprising obtaining a first sample at a first time point and a second sample at a second time point, wherein the second time point is a time after treatment with the compound; contacting the first sample and the second sample with the fluorescent polypeptide of any one of the aspects or embodiments herein; and measuring the amount of fluorescence from said fluorescent polypeptide from the first sample and the second sample,

wherein an increase is amount of fluorescence in the second sample compared to the first sample indicates a compound that has kinase activity.

Other embodiments of the present disclosure are provided infra.

### BRIEF DESCRIPTION OF THE DRAWINGS

FIG. 1A-D is a schematic that shows the features of Phosflour. FIG. 1A and FIG. 1B show the predicted 3D structure of the evolved PhosFluor molecule. FIG. 1C shows a top-down view of FIG. 1B. FIG. 1D shows that kinases recognize phosphorylation recognition sites, which permit engineering of the molecule into a sensor for that kinase.

FIG. 2A-F shows performance of the PKA-PhosFluor biosensor. FIG. 2A shows recombinant PKA-PhosFluor incubated in the absence (control) or presence (PKA) of recombinant catalytic PKA (cat_PKA). FIG. 2B shows IPTG induces expression of T7 RNA polymerase, which transcribes cat_PKA, which phosphorylates PKA-PhosFluor. PKA-PhosFluor and cat_PKA are transcribed from different plasmids maintained by separate antibiotic resistance genes. FIG. 2C and FIG. 2D show charcoal agar plates streaked with E. coli containing the two plasmids indicated in B. The control plate lacks IPTG (FIG. 2C) while the IPTG plate was treated with 1 mM IPTG for 24 hours (FIG. 2D). FIG. 2E shows results of experiments in which HEK-293 cells were grown in 96-well fluorescence plates, transfected with PKA-PhosFluor, and treated with the indicated drug. Graphs depict % fluorescence change (excitation 488 nm; emission 530 nm) measured using a microplate reader 6 hr after addition of the indicated drug. Error bars indicate ±SEM (n=6). FIG. 2F is a schematic diagram showing mechanism of action for each drug.

FIG. 3A-C shows src-PhosFluor functions as a biosensor for Src activity. FIG. 3A shows phase contrast and corresponding fluorescence images of HEK-293 cells transfected with the indicated construct. FIG. 3B shows fluorescence images of HEK-293 cells transfected with Src-PhosFluor, captured at the indicated minutes after 4 mM H2O2 addition. FIG. 3C is a graph that depicts relative fluorescence over time with H2O2 in HEK-293 cells transfected with the indicated construct.

FIGS. 4A-4C show cdk-PhosFluor (nuc) detects cdk2 activity in synchronized cells released into S-phase. FIG. 4A shows images that depict expression of the indicated construct in HEK-293 cells 48-72 hours post-transfection. The c-myc nuclear localization signal (Ray et al., 2015) was attached to PKA-PhosFluor [PKA (nuc)] and cdk-PhosFluor [cdk (nuc)]. FIG. 4B shows a schematic of the cell cycle. Cells can be synchronized at G1/S by incubation with aphidicolin or thymidine for 24 hours. FIG. 4C shows results of experiments where PKA-PhosFluor (nuc) and cdk-PhosFluor (nuc) were transfected into HEK-293 cells. The cells were synchronized and released from G1/S by washing out aphidicolin, and fluorescence images captured. Relative fluorescence from the indicated transfected cells is plotted.

FIG. 5 is a schematic that shows various protein kinases that play a role in cancer.

### DETAILED DESCRIPTION OF THE INVENTION

The present disclosure provides engineered fluorescent proteins (PhosFluor) that displays increased brightness in response to phosphorylation, and uses thereof.

### Definitions

In order that the present disclosure may be more readily understood, certain terms are first defined. In addition, it should be noted that whenever a value or range of values of a parameter are recited, it is intended that values and ranges intermediate to the recited values are also intended to be part of this disclosure. Unless otherwise clear from context, all numerical values provided herein can be modified by the term about.

As used in this specification and the appended claims, the singular forms “a”, “an” and “the” include plural references unless the content clearly dictates otherwise.

The use of the alternative (e.g., “or”) should be understood to mean either one, both, or any combination thereof of the alternatives.

As used herein, the term “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. As used herein, any concentration range, percentage range, ratio range, or integer range is to be understood to include the value of any integer within the recited range and, when appropriate, fractions thereof (such as one tenth and one hundredth of an integer), unless otherwise indicated. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, preferred materials and methods are described herein.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof. Any compositions or methods provided herein can be combined with one or more of any of the other compositions and methods provided herein.

The term “cancer” as used herein refers to the physiological condition in multicellular eukaryotes that is typically characterized by unregulated cell proliferation and malignancy. Cancer includes a variety of cancer types which are well known in the art, including but not limited to, dysplasias, hyperplasias, solid tumors and hematopoietic cancers. Many types of cancers are known to metastasize and shed circulating tumor cells or be metastatic, for example, a secondary cancer resulting from a primary cancer that has metastasized. Additional cancers may include, but are not limited to, the following organs or systems: brain, cardiac, lung, gastrointestinal, genitourinary tract, liver, bone, nervous system, gynecological, hematologic, skin, breast, and adrenal glands. Additional types of cancer cells include gliomas (Schwannoma, glioblastoma, astrocytoma), neuroblastoma, pheochromocytoma, paraganlioma, meningioma, adrenalcortical carcinoma, medulloblastoma, rhabdomyoscarcoma, kidney cancer, vascular cancer of various types, osteoblastic osteocarcinoma, prostate cancer, ovarian cancer, uterine leiomyomas, salivary gland cancer, choroid plexus carcinoma, mammary cancer, pancreatic cancer, colon cancer, and megakaryoblastic leukemia; and skin cancers including malignant melanoma, basal cell carcinoma, squamous cell carcinoma, Karposi's sarcoma, moles dysplastic nevi, lipoma, angioma, dermatofibroma, keloids, sarcomas such as fibrosarcoma or hemangiosarcoma, and melanoma.

The term “operably linked,” as used herein, refers to that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

The term “increase” (and like terms) is used herein to generally refer to the act of improving or increasing, either directly or indirectly, a concentration, level, function, activity, or behavior relative to the natural, expected, or average, or relative to a control condition.

The term “decrease” (and like terms) is used herein to generally refer to the act of reducing, either directly or indirectly, a concentration, level, function, activity, or behavior relative to the natural, expected, or average, or relative to a control condition.

The term “kinase recognition sequence or motif” is used herein to refer to any structure or sequence that is recognized by an enzyme that directs or helps in the enzymatic modification of the substrate by the enzyme. The kinase recognition motif can be within, close to, or part of the structure, such as amino acid residue or residues, that are modified by the activity, such as an enzyme activity, (such as the substrate site for an activity). For example, the sequence surrounding a protein kinase A phosphorylation site plays a significant role in controlling how efficiently the site is modified. Also, protein-protein interaction domains and protein localization domains can control the efficiency of enzymatic modifications of a substrate, such as a protein substrate, and are particularly important within cells (see, Pawson et al., Science 278:2075-2080 (1997). These protein-protein interaction domains and protein localization domains can be distal from the substrate recognition motif and play a role in substrate recognition.

The term “promoter,” as used herein refers to a region or regions of a nucleic acid sequence that regulates transcription.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well. According to some embodiments, the peptide is of any length or size.

The term “nucleic acid molecule” as used herein refers to a single or double-stranded polymer of deoxyribonucleotide or ribonucleotide bases. It includes chromosomal DNA and self-replicating plasmids, vectors, mRNA, tRNA, siRNA, etc. which may be recombinant and from which exogenous polypeptides may be expressed when the nucleic acid is introduced into a cell.

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity.” (a) The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence. (b) The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches. Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences, 8:155-65 (1992), and Pearson, et al., Methods in Molecular Biology, 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995). Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915). In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs may be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters may be employed alone or in combination. (c) The term “sequence identity” or “identity” in the context of two nucleic acid or polypeptide sequences is used herein to refer to the residues in the two sequences that are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA). (d) The term “percentage of sequence identity” is used herein mean the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. (e) The term “substantial identity” of polynucleotide sequences means that a polynucleotide comprises a sequence that has at least 70% sequence identity, at least 80% sequence identity, at least 90% sequence identity and at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid. Mutations may also be made to the nucleotide sequences of the present proteins by reference to the genetic code, including taking into account codon degeneracy.

Amino acid substitutions, insertions, deletions, and other changes in amino acid sequence with respect to a parent polypeptide's amino acid sequence are typically indicated by amino acid number. These and other modifications are defined herein with reference to the amino acid sequence; the first amino acid identified is the one found at the indicated location in the parent sequence, while the second indicates the substitution found in the modified form (e.g., “A71G” indicates a substitution of the amino acid alanine at position 71 in the parent sequence by glycine at the position corresponding to 71 in the variant sequence).

The term “fluorescence” as used herein refers to the molar extinction coefficient at an appropriate excitation wavelength, the fluorescent quantum efficiency, the shape of the excitation spectrum or emission spectrum, the excitation wavelength maximum or emission wavelength maximum, the ratio of excitation amplitudes at two different wavelengths, the ratio of emission amplitudes at two different wavelengths, the excited state lifetime, the fluorescent anisotropy or any other measurable property of a fluorescent compound. A measurable difference in any one of these properties suffices for the utility of the fluorescent compounds of the invention. A difference in fluorescence of a fluorescent compound can be measured by determining the amount of any quantitative fluorescent property, e.g., the amount of fluorescence at a particular wavelength, or the integral of fluorescence of the emission spectrum. Determining ratios of excitation amplitude or emission amplitude at two different wavelengths (“excitation amplitude ratioing” and “emission amplitude ratioing,” respectively) are particularly advantageous because the ratioing process provides an internal reference an cancels out variations in the absolute brightness of the excitation source, the sensitivity of the detector, and light scattering or quenching by the sample.

The term “fluorescent protein” as used herein refers to any protein or fragment thereof capable of fluorescence when excited with appropriate electromagnetic radiation. This includes fluorescent proteins whose amino acid sequences are either naturally occurring or engineered (i.e., analogs) and proteins that have been modified to be fluorescent, such as by the addition of a fluorescence compound, such as fluorescein, rhodamine, Cy3-5, Cy-PE, lucifer yellow, C6-NBD, Dio-Cn(3), FITC, Biodipy-FL, eosin, propidium iodide, tetramethyl rhodamine B, Dil-Cn-(3), Lissamine Rhodamine B, Texas Red, Allophycocyanin, Dil-Cy-5, and squaranes by methods known in the art. For fluorescent compounds, see Molecular Probes Catalogue (1998), U.S. Pat. No. 5,631,169, issued May 20, 1997, U.S. Pat. No. 5,145,774, issued Sep. 8, 1992, and world wide web site http://optics.jct.ac.il/-aryeh/Confocal/fluoreochromes (Jul. 6, 1998) Many cnidarians use green fluorescent proteins (“GFPs”) as energy-transfer acceptors in bioluminescence. A “green fluorescent protein,” as used herein, is a protein that fluoresces green light. Similarly, “blue fluorescent proteins” fluoresce blue light and “red fluorescent proteins” fluoresce red light. GFPs have been isolated from the Pacific Northwest jellyfish, Aequorea victoria, the sea pansy, Renilla reniformis, and Phialidium gregarium (W. W. Ward et al., Photochem. Photoobiol, 35:803-808 (1982); Levine et al, Comp. Biochem. Physiol., 72B:77-85 (1982); and Roth, Purification and Protease Susceptibility of the Green-Fluorescent Protein of Aequorea Aequorea With a Note on Halistaura. Dissertation, Rutgers, The State University of New Jersey, New Brunswick, N.J. (1985)). GFPs have also been engineered to be blue fluorescent proteins and yellow fluorescent proteins (U.S. Pat. No. 5,625,048 to Tsien et al., issued Apr. 29, 1997; WO 97/28261 to Tsien et al., filed Jul. 16, 1997; PCT/US 97/14593 to Tsien et al., filed Aug. 15, 1997: WO 97/28261 to Tsien, published Aug. 7, 1997; and WO 96/23810 to Tsien et al., published Aug. 18, 1996).

The term “sample” as used herein refers to any sample suitable for the methods provided by the present invention. Sources of samples include whole blood, tumor biopsies, tissue samples, bone marrow, pleural fluid, peritoneal fluid, central spinal fluid, urine, saliva and bronchial washes.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “variant” as used herein refers to a polypeptide which differs from the original protein by one or more amino acid substitutions, deletions, insertions, or other modifications. These modifications do not significantly change the biological activity of the original protein. In many cases, a variant retains at least 10%, 20%, 30%, 40%, 50%, 60%, 70%, 80%, 90%, 95%, or 100% of the biological activity of original protein. The biological activity of a variant can also be higher than that of the original protein. A variant can be naturally-occurring, such as by allelic variation or polymorphism, or be deliberately engineered.

The amino acid sequence of a variant is substantially identical to that of the original protein. In many embodiments, a variant shares at least 50%, 60%, 70%, 80%, 85%, 90%, 95%, 99%, or more global sequence identity or similarity with the original protein. Sequence identity or similarity can be determined using various methods known in the art, such as Basic Local Alignment Tool (BLAST), dot matrix analysis, or the dynamic programming method. In one example, the sequence identity or similarity is determined by using the Genetics Computer Group (GCG) programs GAP (Needleman-Wunsch algorithm) The amino acid sequences of a variant and the original protein can be substantially identical in one or more regions, but divergent in other regions.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods and materials are now described.

Reference will now be made in detail to preferred embodiments of the disclosure. While the disclosure will be described in conjunction with the preferred embodiments, it will be understood that it is not intended to limit the disclosure to those preferred embodiments. To the contrary, it is intended to cover alternatives, modifications, and equivalents as may be included within the spirit and scope of the disclosure as defined by the appended claims.

### I. Compositions

The present disclosure features engineered fluorescent proteins (PhosFluor) that display increased brightness in response to phosphorylation. According to one aspect, the present disclosure features an engineered fluorescent protein (PhosFluor) that displays increased brightness in response to phosphorylation. The protein was originally derived from Cyphastrea microphthalma, a coral residing on the Great Barrier Reef of Australia, and selected through more than 100 rounds of protein-directed evolution and site-directed mutations. The amino acid sequence of PhosFlour is shown below in SEQ ID NO:1.

SEQ ID NO: 1
MKIKLRMEGDVNGHPFVITGEGSGKPYEGTQTVDLKVKEGGPLPFAYDIL
TVAFQYGNRAFTKYPADIPDYFKQSFPDGYCWERSMVFEDQGSCVVKSVI
SLDKKEPDCFIYDIRFKGKNFPATGPIMQKETVKWDASTQRMYERDGVLV
GDAKMKLKLKGGGHYRVDIKSTYRAKGVVQYMPGNHYVDHHIEILHHDKD
YNSVTVYESAEARHCRPSSKAE

According to a first aspect, the present disclosure features an isolated fluorescent polypeptide (PhosFluor) comprising an amino acid sequence at least 90% identical to SEQ ID NO: 1, or variants thereof.

According to one embodiment, the amino acid sequence is at least 91% identical to SEQ ID NO: 1. According to one embodiment, the amino acid sequence is at least 92% identical to SEQ ID NO: 1. According to one embodiment, the amino acid sequence is at least 93% identical to SEQ ID NO: 1. According to one embodiment, the amino acid sequence is at least 94% identical to SEQ ID NO: 1. According to one embodiment, the amino acid sequence is at least 95% identical to SEQ ID NO: 1. According to one embodiment, the amino acid sequence is at least 96% identical to SEQ ID NO: 1. According to one embodiment, the amino acid sequence is at least 97% identical to SEQ ID NO: 1. According to one embodiment, the amino acid sequence is at least 98% identical to SEQ ID NO: 1. According to one embodiment, the amino acid sequence is at least 99% identical to SEQ ID NO: 1. According to one embodiment, the amino acid sequence corresponds to SEQ ID NO: 1.

Fluorescent polypeptides can have structures that allow them to be altered from a first state to a second state by, for example, binding of a ligand, post translational modifications such as phosphorylation, dephosphorylation, proteolysis, or glycosylation at specific sites. Compounds can be modified to include such specific sites using methods known in the art. According to one embodiment, SEQ ID NO:1 comprises one or more mutations.

When an activity (e.g. kinase activity) can modify a fluorescent compound, the fluorescent compound can comprise a naturally occurring substrate recognition motif (for example, endogenous to the fluorescent compound) for such enzymatic reactions, or such substrate recognition motifs can be added or engineered into the fluorescent compound (for example exogenous to the fluorescent compound). For example, a fluorescent compound that is a protein can be engineered to comprise a substrate recognition motif for a protease, protein phosphatase, protein kinase, protein prenyltransferase, glycosylase, or any other enzyme using methods known in the art. For example, genetic engineering, chemical modification techniques, or enzyme reactions can be used to add such substrate recognition motifs to the amino- or carboxy-terminus of a fluorescent compound Alternatively, these techniques can be used to insert such substrate recognition motifs within the structure of the fluorescent compound.

A consensus phosphorylation recognition motif for protein kinase A is RRXSZ or RRXTZ, wherein X is any amino acid and Z is a hydrophobic amino acid, preferably valine, leucine or isoleucine. Many variations in the above sequence are allowed, but generally exhibit poorer kinetics. For example lysine (K) can be substituted for the second arginine. Many consensus sequences for other protein kinases have been tabulated (e.g. by Kemp and Pearson, Trends Biochem. Sci. 15:342-346 (1990); Songyang et al., Current Biology 4:973-982 (1994); Nishikawa et al., J. Biol. Chem. 272952-960 (1997); and Songyang et al., Mol. Cell. Biol. 16:6486-6493 (1996)).

For example, a fluorescent protein substrate selective for phosphorylation by cGMP-dependent protein kinase can include the following consensus phosphorylation recognition motif sequence: BKISASEFDRPLR, where B represents either lysine (K) or arginine (R), and the first S is the site of phosphorylation (Colbran et al, J. Biol. Chem. 267:9589-9594 (1992)). The residues DRPLR are less important than the phenylalanine (F) just preceding them for specific recognition by cGMP-dependent protein kinase in preference to cAMP-dependent protein kinase.

Either synthetic or naturally occurring phosphorylation recognition motifs can be used to create a protein kinase phosphorylation site. For example, peptides including the motif XRXXSXRX, wherein X is any amino acid, are among the best synthetic substrates (Kemp and Pearson, supra) for protein kinase C. Alternatively, the Myristoylated Alanine-Rich Kinase C substrate (“MARCKS”) is one of the best substrates for PKC and is an efficient real target for the kinase in vivo. The Examples set forth additional substrates for PKC. The phosphorylation recognition motif sequence around the phosphorylation site of MARCKS is KKKKRFSFK (Graff et al., J. Biol. Chem. 266:14390-14398 (1991)). Either of these two sequences can be incorporated into a fluorescent protein to make it a substrate for protein kinase C.

A protein substrate for Ca2+/calmodulin-dependent protein kinase I is derived from the sequence of synapsin, a known optimal substrate for this kinase. The phosphorylation recognition motif around the phosphorylation site is LRRLSDSNF (Lee et al., Proc. Natl. Acad. Sci. USA 91:6413-6417 (1994).

A protein substrate selective for Ca2+/calmodulin-dependent protein kinase II is derived from the sequence of glycogen synthase, a known optimal substrate for this kinase. The recognition sequence around the phosphorylation site is KKLNRTLTVA (Stokoe et al. Biochem. J. 296:843-849 (1993)). A small change in this sequence to KKANRTLSVA makes the latter specific for MAP kinase activated protein kinase type 1. One skilled in the art would realize that many proteins that do not contain such preferred phosphorylation motifs and sites can be phosphorylated if they conform to the consensus motif, but that the rates of phosphorylation can be less than for the preferred substrates.

A list of other peptides that can be phosphorylated (and the corresponding kinases) is found in Table I of Pinna & Donella-Deana, Biochimica et Biophysica Acta 1222: 415-431 (1994); incorporated herein by reference in its entirety.

Thus, according to another embodiment, the mutation comprises the addition of a substrate recognition site. According to one embodiment, the protein further comprises at least one kinase recognition sequence. According to another embodiment, the kinase recognition sequence is fused to the N-terminus of SEQ ID NO:1. According to another embodiment, the kinase recognition sequences recognizes protein kinase A (PKA).

The substrate specificities of protein kinases have been found, in many cases, to be determined at least in part by short regions within the substrate known as docking sites. Docking sites are modular and self-contained, that is, they can be attached to different proteins to direct their phosphorylation by a specific kinase. Several docking sites can be present in a single substrate, increasing affinity for a kinase. Exemplary docking sites for various substrates are shown in Table 1 below.

TABLE 1

| Substrate | Docking site | Protein kinase |
|---|---|---|
| Mnk2, Rsk | LAQRRX4L | ERK |
| Mnk1, Msk1 | LA + RR | ERK, p38 |
| LIN-1, Elk-1, SAP- | FXFP | ERK |
| 1a, SAP-2 | | |
| Elk-1 | D-domain | ERK2, JNK |
| | KX2 + X3LXL | |
| Spi-B | 1-108, | ERK, JNK |
| cJun | Delta domain | JNK2 |
| | KX2 + X4LXL | |
| NF-AT4 | A-2 domain | Casein kinase 1α |
| | SDASSCES | |
| p107, p130, p27, | RRLFG | Cyclin D-Cdk4 |
| p57, p21, E2F1 | | (Mrail in cyclin D) |
| MKK4 | 1-87 | MEKK1 |
| MEK1, MEK2 | 270-307proline-rich | Raf-1 |
| | insert | |
| ATF2 | 47-66 | JNK |
| Crk | SH3 domain | Abl(proline-rich |
| | | region) |
| AFAP-110 | Proline-rich regions | SH3-containing |
| | | tyrosine kinases |
| p130Cas | pYXXP | Abl |

Accordingly, in one embodiment, the protein further comprises at least one protein kinase docking site.

Signal sequences are used to direct proteins from the cytosol into the ER, mitochondria, chloroplasts, and peroxisomes, and they are also used to transport proteins from the nucleus to the cytosol and from the Golgi apparatus to the ER. Each signal sequence specifies a particular destination in the cell. Exemplary signal sequences are described, for example, in The Compartmentalization of Cells, Molecular Biology of the Cell. 4th edition. Alberts B, Johnson A, Lewis J, et al. New York: Garland Science; 2002, incorporated by reference in its entirety herein.

According to one embodiment, the protein further comprises at least one targeting sequence that directs the protein to a specific cellular compartment.

A change in the tertiary structure of a fluorescent compound by the addition or removal of a moiety by chemical or enzymatic activity can also cause a change in a fluorescent property of a fluorescent compound after quenching. For example, phosphorylation of a fluorescent compound can lead to a change in its tertiary structure through the creation of new or stronger interactions between amino acid residues, which can result in stabilized fluorescence that can result in increased resistance to quenching. Such a change in sensitivity to quenching can subsequently be used to measure the amount of fluorescent compound that has been phosphorylated, and hence the activity of the kinase can be detected and/or measured. Such moieties can also destabilize the tertiary structure of a fluorescent protein, which can result in destabilized fluorescence under quenching conditions. Furthermore, enzymatic activities such as proteases can alter the tertiary structure of a fluorescent protein, which can also result in destabilized fluorescence under quenching conditions. Furthermore, the presence of an electrochemical, chemical or electrical gradient or potential can also change a fluorescent property of a fluorescent compound that can be detected through quenching.

In one embodiment, the fluorescent protein contains a phosphorylation motif and site at or about one or more of the termini, in particular, the amino-terminus, of the fluorescent protein. The site preferably is located in a position within five, ten, fifteen, or twenty amino acids of a position corresponding to the wild type amino-terminal amino acid of the fluorescent protein moiety. This includes sites engineered into the existing amino acid sequence of the fluorescent protein moiety and can also be produced by extending the amino terminus of the fluorescent protein moiety. According to one embodiment, the kinase recognition sequence is fused to the N-terminus of SEQ ID NO:1.

Fluorescent proteins having a phosphorylation site at or about a terminus of a fluorescent protein moiety offer, but are not limited to, the following advantages. First, it is often desirable to append additional amino acid residues onto the fluorescent protein moiety to create a specific phosphorylation consensus sequence. Such a sequence is less likely to disrupt the folding pattern of a fluorescent protein moiety when appended onto the terminus than when inserted into the interior of the protein sequence. Second, different phosphorylation motifs can be interchanged without significant disruption of the fluorescent protein, thereby providing a general method of measuring different kinases. Third, the phosphorylation site is preferably exposed to the surface of the protein and, therefore, more accessible to protein kinases.

In addition, the kinetics of phosphorylation of the described fluorescent proteins can be enhanced. For example, the efficiency with which a phosphorylation site is modified by a kinase or phosphatase is dependent on the sequence and accessibility of the recognition motif. The accessibility of the phosphorylation motif can be improved my making changes in amino acids that disorder the local amino-terminal structure or reduce interactions between the amino-terminal region and the interior of the molecule.

According to one embodiment of the above aspects and embodiments, the isolated fluorescent polypeptide further comprises a second fluorescent molecule. According to one embodiment, the second fluorescent molecule is a fluorescent protein or a quantum dot.

According to one embodiment, the present disclosure features a nucleic acid encoding the isolated fluorescent polypeptide of any one of the above aspects or embodiments. According to one embodiment, the present disclosure features a cell comprising the nucleic acid.

While certain florescent compounds can be prepared chemically, for example, by coupling a fluorescent moiety to the amino terminus of a protein moiety, in certain embodiments it is preferable to produce fluorescent compounds comprising a peptide or protein recombinantly.

Recombinant production of a fluorescent compound involves expressing a nucleic acid molecule having sequences that encode a peptide or protein. A nucleic acid molecule includes both DNA and RNA molecules. It will be understood that when a nucleic acid molecule is said to have a DNA sequence, this also includes RNA molecules having the corresponding RNA sequence in which “U” replaces “T.” The term “recombinant nucleic acid molecule” refers to a nucleic acid molecule which is not naturally occurring, and which comprises two nucleotide sequences that are not naturally joined together. Recombinant nucleic acid molecules are produced by artificial combination, e.g., genetic engineering techniques or chemical synthesis.

In one embodiment, the nucleic acid encodes an isolated fluorescent polypeptide in which a single polypeptide includes the fluorescent protein moiety within a longer polypeptide. In another embodiment, the nucleic acid encodes an amino acid sequence that comprises a substrate site for an activity consisting essentially of a fluorescent protein moiety modified to include a substrate site for an activity. In either case, nucleic acids that encode fluorescent proteins are useful as starting materials.

Mutant versions of fluorescent proteins can be made by site-specific mutagenesis of other nucleic acids encoding a fluorescent protein moiety or by random mutagenesis caused by increasing the error rate of PCR of the original polynucleotide with 0.1 mM MnCl2 and unbalanced nucleotide concentrations (U.S. Pat. No. 5,625,048 to Tsien, issued Apr. 29, 1997; and PCT/US95/14692, filed Nov. 10, 1995).

Nucleic acids encoding fluorescent compounds that are fusions between, e.g., a polypeptide including a phosphorylation site and a fluorescent protein moiety, a protein kinase docking site or a targeting sequence that directs the protein to a specific cellular compartment can be made by ligating nucleic acids that encode each of these. Nucleic acids encoding fluorescent compounds that include the amino acid sequence of a fluorescent protein moiety in which one or more amino acids in the amino acid sequence of a fluorescent protein moiety are substituted to create a substrate site for an activity can be created by, for example, site specific mutagenesis of a nucleic acid encoding a fluorescent protein moiety.

Nucleic acids used to transfect cells with sequences coding for expression of a polypeptide of interest such as those encoding a fluorescent compound generally will be in the form of an expression vector including expression control sequences operatively linked to a nucleotide sequence coding for expression of the polypeptide. As used herein, the term “nucleotide sequence coding for expression of a polypeptide” refers to a sequence that, upon transcription and translation of mRNA, produces the polypeptide. As any person skilled in the art recognizes, this includes all degenerate nucleic acid sequences encoding the same amino acid sequence. This can include sequences containing, e.g. introns. As used herein, the term “expression control sequences” refers to nucleic acid sequences that regulate the expression of a nucleic acid sequence to which it is operatively linked. Expression control sequences are “operatively linked” to a nucleic acid sequence when the expression control sequence control and regulate the transcription and, as appropriate, translation of the nucleic acid sequence. Thus, expression control sequences can include appropriate promoters, enhancers, transcription termination, or a start codon, (i.e., ATG) in front of a protein-encoding gene, splicing signals for introns, maintenance of the correct reading frame of that gene to permit proper translation of the mRNA, and stop codons. Recombinant nucleic acid can be incorporated into an expression vector comprising expression control sequences operatively linked to the recombinant eukaryotes by inclusion of appropriate promoters, replication sequences, market, etc. The expression vector can be transfected into a host cell for expression of the recombinant nucleic acid. Host cells can be selected for high levels of expression in order to purify the protein. E. coli is useful for this purpose. Alternatively, the host cell can be a prokaryotic or eukaryotic cell selected to study the activity of an enzyme produced by the cell. The cell can be, e.g. a cultured cell or a cell in vivo. The construction of expression vectors and the expression of genes in transfected cells involves the use of molecular cloning techniques also well known in the art (Sambrook et al., Molecular Cloning—A Laboratory Manual, Cold Spring Harbor Laboratory, Cold Spring Harbor, N.Y. (1989); Ausubel et al., Current Protocols in Molecular Biology, Greene Publishing Associates and John Wiley & Sons, Inc.).

Recombinant fluorescent protein substrates can be produced by expression of nucleic acid encoding for the protein in E. coli.

The construct can also contain a tag to simplify isolation of the expressed fluorescent compound. For example, a polyhistidine tag of, e.g. six histidine residues, can be incorporated at the amino or carboxyl terminal of the fluorescent compound. The polyhistidine tag allows convenient isolation of the protein in a single step by nickel chromatography.

Alternatively, the fluorescent compound, such as a fluorescent protein substrate, need not be isolated from the host cells. This method is particularly advantageous for the assaying for the presence of an activity in situ.

The compositions may be pharmaceutical compositions.

The present disclosure encompasses the preparation and use of pharmaceutical compositions comprising a fluorescent polypeptide of the disclosure as an active ingredient. Such a pharmaceutical composition may consist of the active ingredient alone, as a combination of at least one active ingredient, or the pharmaceutical composition may comprise the active ingredient and one or more pharmaceutically acceptable carriers, one or more additional (active and/or inactive) ingredients, or some combination of these.

As used herein, the term “pharmaceutically acceptable carrier” means a chemical composition with which the active ingredient may be combined and which, following the combination, can be used to administer the active ingredient to a subject.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

### II. Methods

The present invention includes methods for determining whether a sample contains an activity using a fluorescent polypeptide of the present invention. Depending on the type of activity to be determined, different fluorescent compounds are to be used. For example, if a protein kinase activity is to be determined, then a fluorescent compound that is a substrate for a protein kinase is used in the present invention.

As is known in the art, different cofactors are required for different enzyme reactions. Thus, in certain embodiments, such cofactors should be present in the assay conditions for those enzymes. For example, protein kinases add a phosphate residue to the phosphorylation site of a protein generally through the hydrolysis of ATP to ADP. Fluorescent compounds that are substrates for protein kinases are useful in assays to determine the amount of protein kinase activity in a sample.

Accordingly, in one another aspect, the present disclosure features a method for determining whether a sample contains an activity, comprising obtaining a sample; contacting the sample with the fluorescent polypeptide of any one of the aspects and embodiment herein; and measuring the amount of fluorescence from said fluorescent polypeptide. According to one embodiment, the activity is kinase activity. According to one embodiment, the activity is phosphatase activity. According to another embodiment, the method further comprises the step of comparing the amount of fluorescence measured from said fluorescent polypeptide with the amount of fluorescence from a control sample. A control sample can be a sample that does not contain the activity (e.g. kinase activity), or contains a known amount of activity (e.g. kinase activity). For example, comparisons can be made with a control sample known not to contain an activity, a control sample known to contain an activity (preferably in a known amount), a control sample representing background signal, or a control sample with or without test compounds.

In one embodiment, the amount of an activity in the sample can be calculated as a function of the difference in the determined amount of quenching at the two times. For example, the absolute amount of an activity can be calibrated using standards of activity determined for certain amounts of activity after certain amounts of time. The faster or larger the difference in the amount of quenching, the more activity is present in the sample.

In another embodiment, fluorescence in a sample is measured using a fluorimeter. In general, excitation radiation from an excitation source having a first wavelength, passes through excitation optics. The excitation optics causes the excitation radiation to excite the sample. In response, fluorescent compounds in the sample emit radiation that has a wavelength that is different from the excitation wavelength. Collection optics then collect the emission from the sample. The device can include a temperature controller to maintain the sample at a specific temperature while it is being scanned. Methods of performing assays on fluorescent materials are well known in the art. (Lakowics, Principles of Fluorescence Spectroscopy, Plenum Press, N Y (1983); Herman, Fluorescence Microscopy of Living Cells in Culture, Part B, Methods in Cell Biology, volume 30, Academic Press, San Diego, pp. 219-243 (1989); Turro, Modern Molecular Photochemistry, Menlo Park, Calif., Benjamin/Cummings Publishing, pp. 296-361 (1978)). In one embodiment, a cell is transiently or stably transfected with an expression vector encoding a fluorescent polypeptide described herein containing a substrate site for an activity to be assayed. This expression vector optionally includes controlling nucleotide sequences such as promoter or enhancing elements. The expression vector expresses the fluorescent compound that contains the substrate site for an activity to be detected. The activity to be assayed may either be intrinsic to the cell or may be introduced by stable transfection or transient co-transfection with another expression vector encoding the activity and optionally including controlling nucleotide sequences such as promoter or enhancer elements. The fluorescent compound and the activity preferably are located in the same cellular compartment so that they have more opportunity to come into contact. Membrane-bound or membrane-associated fluorescent compounds can also be used in this and any other method of the present invention. The amount of activity is then determined by measuring the fluorescence of the sample (which can contain whole cells) under quenching conditions, and comparison to appropriate controls, such as controls that either do not contain the activity, or contain a known amount of activity.

The sample can be any sample, such as a sample of cells, tissue, organ, or fluid obtained from an organism (such as a mammalian, such as a human) or an extract obtained therefrom. Miniaturized arrays of samples attached to a matrix, such as a bead or solid support as they are known in the art or later developed, can be used in the present invention to detect fluorescence or other activity in a sample. A sample can also comprise cultured cells, culture fluid, or extracts or conditioned media obtained therefrom. The cells can be prokaryotic or eukaryotic, such as mammalian cells, such as human cells. According to one embodiment, the sample is selected from the group consisting of: a blood sample, a tissue sample or a tumor biopsy. According to one embodiment, the sample is from a subject suffering from a disease or disorder. According to another embodiment, the disease or disorder is cancer. According to one embodiment, measuring the amount of fluorescence is used to determine changes in subcellular localization, relative abundance or activation kinetics. According to one embodiment, measuring the amount of fluorescence is used to determine risk of metastasis or resistance to therapeutic agents.

The methods of the invention can be used in drug screening to determine whether a test compound alters an activity. In one embodiment, the assay is performed on a sample in vitro suspected of containing an activity.

According to another aspect, the present disclosure features a method of screening for a compound that has kinase activity, comprising obtaining a first sample at a first time point and a second sample at a second time point, wherein the second time point is a time after treatment with the compound; contacting the first sample and the second sample with the fluorescent polypeptide of any one of the aspects or embodiments herein; and measuring the amount of fluorescence from said fluorescent polypeptide from the first sample and the second sample, wherein an increase is amount of fluorescence in the second sample compared to the first sample indicates a compound that has kinase activity.

In one embodiment, the first sample is a sample containing a known amount of activity that is contacted with the fluorescent polypeptide of the invention and a test compound.

Libraries of host cells expressing fluorescent polypeptides as described herein are useful in identifying fluorescent proteins having peptide moieties that exhibit quenching. Several methods of using the libraries are envisioned. In general, one begins with a library of recombinant host cells, each of which expresses a different fluorescent compound, such as fluorescent compound comprising a protein, peptide, or nucleic acid. Each cell is expanded into a clonal population that is genetically homogeneous.

In a first method, fluorescence quenching is measured or compared from each clonal population before and after at least one specified time after a known change in an intracellular activity. Alternatively, fluorescence quenching measured in each clonal population can be compared with the results obtained using untreated control cells. For example, a change in kinase activity could be produced by transfection with a gene encoding a kinase activity, by increasing the expression of the kinase using expression control elements, or by any condition that post-translationally modulates the kinase activity. Examples of the latter include cell surface receptor mediated elevation of intracellular cAMP to activate cAMP-dependent kinases, surface receptor mediated increases of intracellular cGMP to activate cGMP-dependent protein kinase, increases in cytosolic free calcium to activate Ca2+/calmodulin-dependent protein kinase types I, II, or IV, or the production of diacylglycerol to activate protein kinase C, etc. One then selects for the clone(s) that show the largest or fastest changes in fluorescence in response to quenching compared to non-treated control cells.

The present invention also includes a compound identified by any method of the present invention. Such compounds can be provided as a pharmaceutical composition in a pharmaceutically acceptable carrier as is set forth in U.S. patent application Ser. No. 09/030,578, filed Feb. 24, 1998. The present invention also includes a library of such compounds, which comprise two or more of such compounds provided either separately or in combination. The present invention also includes a system used to screen and identify compounds, such as set forth in U.S. patent application Ser. No. 08/858,016, filed May 16, 1997.

In another embodiment, the ability of a compound to alter an activity in vivo is determined. In an in vivo assay, cells transfected with an expression vector encoding a fluorescent compound, such as a fluorescent polypeptide, of the invention are exposed to at least one amount of at least one test compound, and the fluorescence after quenching in each cell (individually or as a population) can be determined. Typically, the difference is calibrated against standard measurements (for example, in the presence or absence of test compounds) to yield an absolute amount of activity. A test compound that inhibits or blocks the activity or expression of an activity can be detected by a relative change in fluorescence after quenching. The cell can also be transfected with an expression vector to coexpress the activity or an upstream signaling component such as a receptor, and the fluorescent polypeptide. This method is useful for detecting signaling to an activity such as a protein kinase of interest from an upstream component of a signaling pathway. If a signal from an upstream molecule, for example a receptor (preferably in the presence of an agonist), is inhibited by a test compound, then the kinase activity will be inhibited as compared to controls incubated without the test compound. This provides a method for screening for compounds that affect cellular events (including receptor-ligand binding, protein-protein interaction, or kinase activation), and signal to the target kinase. This method can use cultured cells or extracts or conditioned media derived therefrom. This method can also use cells derived from an organism, such as a mammal, such as a human. Such cells can be derived from a tissue, organ or fluid. The sample can also comprise an extract of such cells.

### III. Kits

This disclosure also provides kits containing a fluorescent polypeptide as described herein, and optionally cofactors for an activity. In one embodiment, the kit comprises at least one container holding the fluorescent polypeptide and optionally a second container holding a cofactor or buffer. Optionally, the kit can comprise other reagents or labware to practice a method, such as a method of the present invention. The entire kit can be provided in a separate container, such as a box. This container can include instructions for use of the contents in a method of the present invention, or for other purposes.

A number of embodiments of the disclosure have been described. Nevertheless, one skilled in the art, without departing from the spirit and scope of the disclosure, can make various changes and modifications of the disclosure to adapt it to various usages and conditions. Accordingly, the following examples are intended to illustrate, but not limit, the scope of the invention claimed.

### EXAMPLES

### Example 1. PhosFlour Biosensors

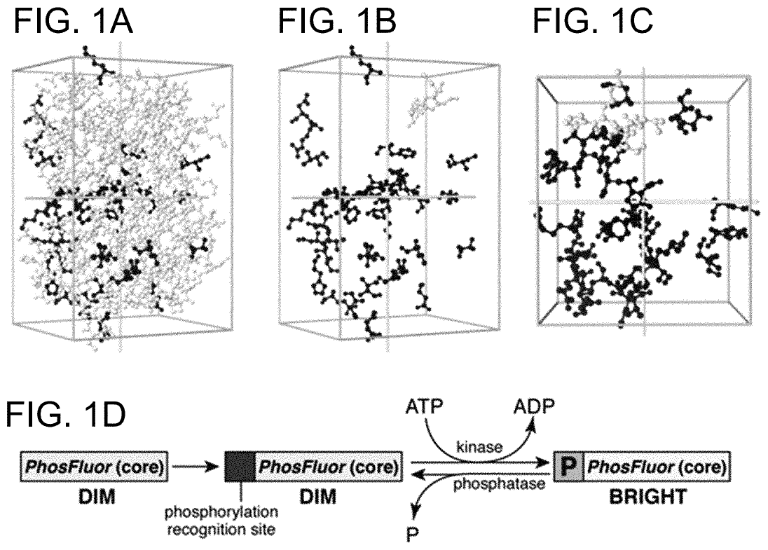

PhosFluor is a unique proprietary protein derived from coral residing in Australia's Great Barrier Reef. The original protein was extensively optimized by protein-directed evolution and site-directed mutagenesis, and the evolved, monomeric protein displays exceptional fluorescence sensitivity to N-terminal phosphorylation (FIG. 1). The amino acid sequence of PhosFlour is shown in SEQ ID NO: 1. FIG. 1A-D shows the features of Phosflour. FIG. 1A and FIG. 1B show the predicted 3D structure of the evolved PhosFluor molecule, showing site-directed mutations (red), evolved mutations (blue), the chromophore (black), N-terminus (yellow), and C-terminus (brown). Non-mutated residues (white) are shown in FIG. 1A and removed in FIG. 1B. FIG. 1C shows a top-down view of FIG. 1B. FIG. 1D shows that kinases recognize phosphorylation recognition sites, which permit engineering of the molecule into a sensor for that kinase. Transfer of phosphate from ATP to the protein confers a strong negative charge to the protein, resulting in an increase in fluorescence. Note the proximity of the N-terminus (yellow) to the chromophore (black), which may explain why the molecule responds well to phosphorylation. PhosFluor-based sensors will be capable of monitoring kinases, which add phosphates, and phosphatases, which remove phosphates. Under basal conditions, PhosFluor is essentially quenched, but when phosphorylated, demonstrates increased fluorescence. Substrate recognition sites can be engineered into PhosFluor to create different versions that respond to the activity of selective protein kinases. As proof-of-concept, preliminary data is shown for three PhosFluor-based biosensors engineered to detect the activity of protein kinase A (PKA), Src, and cyclin-dependent kinase (cdk).

### PKA-PhosFluor Biosensor

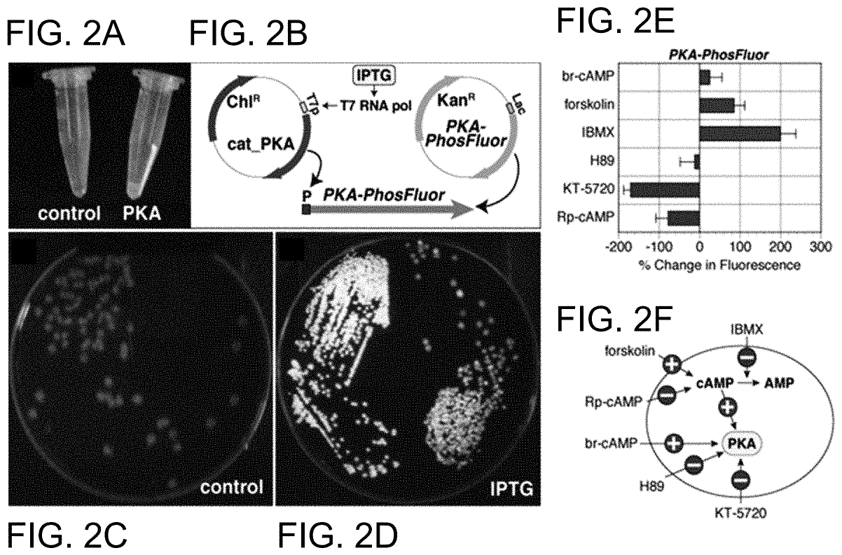

The PKA-PhosFluor biosensor was created by fusing a phosphorylation recognition site for PKA to the N-terminus of PhosFluor. Recombinant PKA-PhosFluor increased its fluorescence when phosphorylated by the catalytic subunit of PKA in a test tube (FIG. 2A). The catalytic subunit does not require activation by cAMP and is capable of phosphorylating PKA substrates alone (Uhler et al., 1986). PKA-PhosFluor was then expressed in bacteria that co-expressed the catalytic subunit of PKA under controllable conditions (FIG. 2B). Without phosphorylation, PKA-PhosFluor barely showed fluorescence (FIG. 2C). When PKA was turned on using IPTG, a marked increase in fluorescence was observed (FIG. 2D). In this system, PKA-PhosFluor behaved like an optical “molecular switch”. These findings indicate that attachment of a PKA substrate recognition site to PhosFluor converts the molecule into a biosensor for PKA activity.

Next, the ability of PKA-PhosFluor to monitor PKA activity in mammalian cells was examined. PKA activity is regulated by the level of cAMP, which itself, is regulated by other signaling pathways and cell growth (Uhler et al., 1986). Three known activators (br-cAMP, forskolin and IBMX) all induced fluorescence of PKA-PhosFluor (FIG. 2E). Three known inhibitors of PKA (H89, KT-5720 and Rp-cAMP) all reduced fluorescence, consistent with their mechanism of action (FIG. 2E-F). All the drugs used have widely varying mechanisms of action (FIG. 2F). These results suggest that PKA-PhosFluor acts as faithful monitor of PKA activity in HEK-293 cells, and has the potential for evaluating tumor responsiveness to kinase inhibitors.

### Src-PhosFluor Biosensor

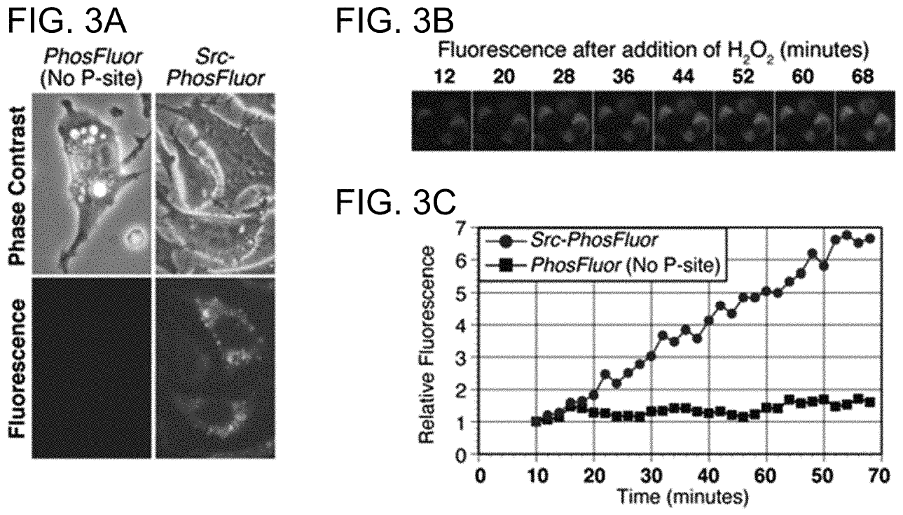

As a second prototype, PhosFluor was converted into a biosensor for Src (Src-PhosFluor). HEK-293 cells expressing PhosFluor lacking phosphorylation sites (No P-site) showed no fluorescence while cells expressing Src-PhosFluor showed fluorescence localized to perinuclear vesicles (FIG. 3A). Src is localized to perinuclear vesicles (Redmond et al., 1992), indicating that the biosensor detects Src activity in the appropriate intracellular locations. A well-established paradigm was then used for inducing Src activity: oxidative stress (Saito et al., 2002; Thakali et al., 2007). A robust increase in fluorescence was observed when HEK-293 cells expressing Src-PhosFluor were treated with H₂O₂, consistent with previous observations (FIG. 3B-C). By contrast, little change was observed in the fluorescence of PhosFluor containing no phosphorylation sites (FIG. 3C). These results indicate that the PhosFluor system is capable of monitoring kinase activity continually in living cells.

### Cdk-PhosFluor Biosensor

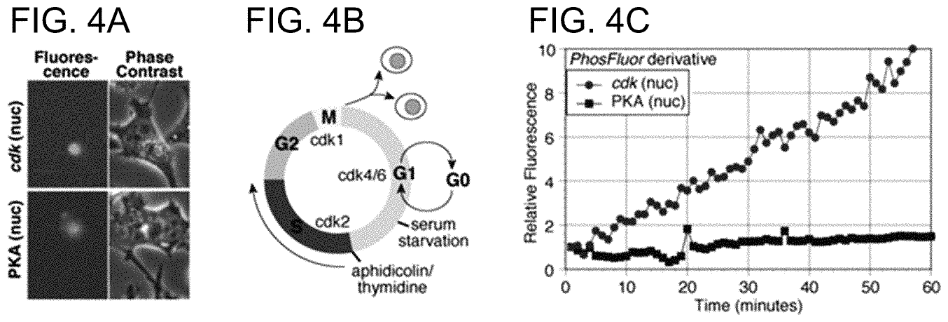

As a third prototype, a phosphorylation recognition site for cyclin-dependent protein kinase (cdk) was fused to the N-terminus of PhosFluor. Cdks regulate different checkpoints throughout the cell cycle by promoting phosphorylation of substrates in the nucleus. Therefore, the c-myc nuclear localization signal was attached to the C-terminus of cdk-PhosFluor and PKA-PhosFluor (as a negative control) (FIG. 4A). HEK-293 cells were synchronized using aphidicolin, a DNA polymerase inhibitor, to ensure that virtually all cells are at the G1/S phase of the cell cycle (Pedrali-Noy et al., 1980) (FIG. 4B). When cells are released from the G1/S block, they proceed into S-phase where cdk2 is the primary driver (Dulic et al., 1992; Koff et al., 1992; Pagano et al., 1993). Cells expressing cdk-PhosFluor (nuc) increased fluorescence as they proceed through S-phase (FIG. 4C), while cells expressing PKA-PhosFluor (nuc) did not. Thus, cdk-PhosFluor (nuc) was capable of detecting cdk2 activity while PKA-PhosFluor (nuc) was not.

These experiments indicate that it should be easy to convert the core PhosFluor molecule into a fluorescent sensor by placing phosphorylation recognition motifs at its N-terminus. Further data (not shown) shows that residues can be inserted into at least two loops that connect the beta strands of PhosFluor, without affecting its function. These structural features will be exploited to optimally design the protein into a specific biosensor for cancer-relevant kinases. Few biological tools are available for revealing the complexity of signaling networks in cancer. Thus, PhosFluor-based biosensors will play a key role in rapidly identifying abnormal cell signaling networks in individual patients.

### Example 2. Engineering Kinase Specificity into PhosFluor-Based Biosensors

Kinases recognize specific motifs surrounding the phosphorylation site on substrate proteins. Many phosphorylation recognition motifs are known, but they are usually not specific enough to convert a protein into a substrate for a single kinase. Additional sequences or docking sites, distal to the phosphorylation site, can confer greater kinase specificity to the substrate.

The core PhosFluor molecule is converted into a fluorescent biosensor by placing phosphorylation recognition motifs at its N-terminus. This is demonstrated for three different kinases: PKA, Src and cdk, as shown in Example 1.

The present experiments show that the biosensors specifically detect the activity of the kinase they were designed to detect, to avoid false-positive signals.

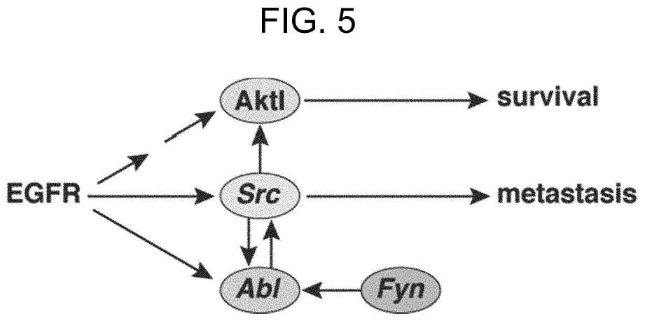

Biosensors specific for Src, Fyn, Abl, and Akt1 will be engineered. The rationale is that all of these kinases play a dominant and well-established role in cancer progression and metastasis (FIG. 5). EGFR (epidermal growth factor receptor) is a receptor tyrosine kinase, and the portal of entry for adeno-associated virus type 6, which we will use to express our biosensors into circulating tumor cells (CTCs). EGFR is amplified in breast and lung cancers, and activates a signaling pathway that promotes tumor proliferation and metastasis. EGFR is undetectable in blood cells. Viral transduction offers a way to selectively express our biosensors in CTCs. Src, Fyn, Abl and Akt1 are protein kinases that have established roles in tumor metastasis and survival. They are all activated downstream of EGFR, and they interact with one another to coordinate their actions. Both Src and Fyn promote loss of cell adhesion and microinvasion through endothelial vessel walls, promote epithelial to mesenchymal transition (which facilitates metastasis), and increases cell motility (reviewed by Patel et al., 2016). Abl or Abl-like kinases are the targets of many kinase inhibitors in use today for cancer treatment (Fabbro et al., 2015). Akt1 is involved in tumor survival and amplified in specific cancers such as colorectal cancer, and mutated in breast and lung cancer. Upregulation of Akt1 is associated with a worse prognosis (Agarwal et al., 2013). All the protein kinases shown are drug targets for cancer treatment. Their activity in CTCs has clinical relevance with respect to prognosis and therapeutic options. Src and Fyn are members of the Src family of kinases, which is comprised of nine members. These experiments focus on breast and lung cancer, so the six Src family members that are primarily expressed in blood cells are not the focus of these experiments. The remaining three members, Src, Fyn and Yes, are expressed in breast and lung tissue, but virtually all Yes substrates are targeted by Src and Fyn (Takeda et al., 2010). Thus, the present experiments will focus on distinguishing between Src and Fyn. Abl is clearly distinct from Src and Akt1 is a Ser/Thr kinase.

Optimal phosphorylation recognition (P-site) and docking motifs have been established for all the kinases that are proposed for study (see Table 2, below). The optimal phosphorylation recognition motifs (P-site) shown in Table 2 were defined using phage display peptide technology. Phosphorylated residues are shown in bold. The SH3 ligand (a docking domain) is located 6-12 residues away from the P-Site in the C-terminal direction. For Src and Fyn, protein sequences for kinase-specific substrates were identified from the dbPTM database, downloaded from Genbank, and SH3 ligand sequences analyzed.

TABLE 2
Optimal phosphorylation and docking
domains motifs of Src, Fyn, Abl, and Akt1.

| Kinase | P-site | Ref | SH3 Ligand | Ref | Notes |
|---|---|---|---|---|---|
| Src | ...EEEIYGEFEAKK | 1 | 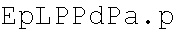 | | SH3 bioinformatics, N = 177 |
| Fyn | ....EgTYGtΩ | 2 | 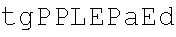 | | SH3 bioinformatics, N = 47 |
| Abl | ......IYA.P | 3 | 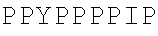 | 5 | Abl-specific |
| | ...AEVIYAAPE | 1 | | | |
| Akt1 | RKRERAYSFGHH | 4 | N/A | | Akt1-specific |

Ω = hydrophobic;
l = Songyang et al., 1995;
2 = Dente et al., 1997;
3 = Till et al., 1999;
4 = Obata et al, 2000;
5 = Rickles et al., 1994, 1995.

Src, Fyn, and Abl are tyrosine kinases, which are organized into SH (Src-homology) domains: SH1 binds to ATP, SH2 binds to phospho-tyrosine, and SH3 binds to specific substrate motifs. Ligands that bind to SH3 domains both activate and confer kinase specificity to the substrate (Moarefi et al., 1997; Scott and Miller, 2000). Both the P-site and SH3 ligand are sufficient for distinguishing phosphorylation by Abl versus Src/Fyn. Peptide display did not reveal substrate sequences in the P-site that distinguish between Src and Fyn. However, bioinformatics did reveal that SH3 ligand sequences differ significantly between Src and Fyn. The Akt1 phosphorylation motif is specific for Akt1, distinguishing it from other Ser/Thr kinases (Obata et al., 2000). Incorporation of these sequences into PhosFluor should convert the molecule into a biosensor specific for the intended kinase (Table 2). The following steps are used to create each biosensor:

(1) Identify the Compatible Internal Regions of PhosFluor that can Accommodate Extra Residues.

All fluorescent proteins conform to a beta-barrel structure with 11 beta-sheets (7-12 residues long) connected by loops to form a beta-barrel (Ormo et al., 1996), including PhosFluor. Structure-function analyses of GFP reveal that beta-sheets cannot be disrupted (Arpino et al., 2014), but at least three loops can accept many residues without affecting its function (Pavoor et al., 2009; Peelle et al., 2001). Preliminary data indicates that PhosFluor can accept additional sequences in at least two loops, between the N- and C-termini; not all positions have been tested yet.

Standard recombinant techniques are used to introduce residues into internal regions of PhosFluor. Expression in bacteria will provide information if the modification is tolerated.

(2) Introduce Phosphorylation Recognition Sites and/or Docking Motifs into the N-Terminal Region and/or Loop Regions of PhosFluor.

Based on data from Step A, and using Table 2, phosphorylation or docking motifs are be inserted into specific regions of the PhosFluor molecule.

### (3) Test the Function of the Biosensor in Bacterial or Human Cell Culture.

The E. coli dual-vector system for expressing two different proteins (Gruber et al., 2008) is a powerful and rapid system to determine if a PhosFluor-based biosensor can detect the activity of a specific protein kinase. E. coli lacks mammalian protein kinases so there is no interference from other kinases in this biological setting. The PhosFluor biosensor and constitutively active forms of the kinase will be expressed in E. coli to make this initial evaluation.

The biosensor is also be expressed in established human cells lines, such as HeLa or HEK-293 cells, for rapid characterization of function. Both HeLa and HEK-293 cells are easily transfected by conventional methods. All Src family kinases are activated by oxidative stress and inhibited by compounds such as PP1 (Thakali et al., 2007). Abl is also activated by oxidative stress (Sun et al., 2000) but is inhibited by Gleevec and related kinase inhibitors (Gross et al., 2015). Akt1 is stimulated by growth factors but can be inhibited by specific pharmacological agents (Agarwal et al., 2013).

### (4) Determine the Specificity of the PhosFluor Biosensor in Human Cell Lines.

To determine if the biosensor functions in a specific manner, the expression of the specific kinase is be knocked down in the human cell line using siRNA and then the biosensor is introduced into that cell. Silencing via siRNA is an established technique for rapidly reducing the expression of a specific gene (Fire et al., 1998). Since this method essentially eliminates the expression of one kinase, the absence of PhosFluor fluorescence under these conditions constitutes rigorous demonstration that the biosensor is kinase-specific in a particular cell line.

A generic Src biosensor and a PKA biosensor (PKA is related to Akt1, also known as PKB) has already been generated. This indicates that creating similar biosensors is highly feasible.

When expressed in human cells, each biosensor should display fluorescence in regions of the cell where activity has been reported at its highest levels. Src and Fyn activity is highest in perinuclear vesicles. Abl is distributed between both the nucleus and cytoplasm, and Akt1 in the cytoplasm. All biosensors should display increased fluorescence when cells are stimulated by serum or growth factors. Silencing using siRNA is a rigorous test of specificity in the cell line to be tested.

It is possible that longer motifs or additional docking sequences are needed to create a more specific biosensor. This will become evident after testing the function and specificity of each biosensor in Steps 3 and 4 above. If so, the desired sequences will be engineered into the molecule and the biosensor will be re-tested.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

### INCORPORATION BY REFERENCE

Each reference, patent and patent application referred to in the instant application is hereby incorporated by reference as if each reference were noted to be incorporated individually.

### REFERENCES

Agarwal, E., Brattain, M. G., and Chowdhury, S. (2013). Cell survival and metastasis regulation by Akt signaling in colorectal cancer. Cell Signal 25, 1711-1719.
Allard, W. J., Matera, J., Miller, M. C., Repollet, M., Connelly, M. C., Rao, C., Tibbe, A. G., Uhr, J. W., and Terstappen, L. W. (2004). Tumor cells circulate in the peripheral blood of all major carcinomas but not in healthy subjects or patients with nonmalignant diseases. Clin Cancer Res 10, 6897-6904.
Arpino, J. A., Reddington, S. C., Halliwell, L. M., Rizkallah, P. J., and Jones, D. D. (2014). Random single amino acid deletion sampling unveils structural tolerance and the benefits of helical registry shift on GFP folding and structure. Structure 22, 889-898.
Ashworth, T. R. (1869). A case of cancer in which cells similar to those in the tumours were seen in the blood after death. Med J Australia 14, 146-147.
Bhagwat, N., and Carpenter, E. L. (2017). Flow Cytometric Methods for Circulating Tumor Cell Isolation and Molecular Analysis. Adv Exp Med Biol 994, 105-118.
Chaffer, C. L., and Weinberg, R. A. (2011). A perspective on cancer cell metastasis. Science 331, 1559-1564.
Cohen, S. J., Punt, C. J., lannotti, N., Saidman, B. H., Sabbath, K. D., Gabrail, N. Y., Picus, J., Morse, M., Mitchell, E., Miller, M. C., Doyle, G. V., Tissing, H., Terstappen, L. W., and Meropol, N. J. (2008). Relationship of circulating tumor cells to tumor response, progression-free survival, and overall survival in patients with metastatic colorectal cancer. J Clin Oncol 26, 3213-3221.
Cristofanilli, M., Budd, G. T., Ellis, M. J., Stopeck, A., Matera, J., Miller, M. C., Reuben, J. M., Doyle, G. V., Allard, W. J., Terstappen, L. W., and Hayes, D. F. (2004). Circulating tumor cells, disease progression, and survival in metastatic breast cancer. N Engl J Med 351, 781-791.
Danila, D. C., Heller, G., Gignac, G. A., Gonzalez-Espinoza, R., Anand, A., Tanaka, E., Lilja, H., Schwartz, L., Larson, S., Fleisher, M., and Scher, H. I. (2007). Circulating tumor cell number and prognosis in progressive castration-resistant prostate cancer. Clin Cancer Res 13, 7053-7058.
Dente, L., Vetriani, C., Zucconi, A., Pelicci, G., Lanfrancone, L., Pelicci, P. G., and Cesareni, G. (1997). Modified phage peptide libraries as a tool to study specificity of phosphorylation and recognition of tyrosine containing peptides. J Mol Biol 269, 694-703.
Dulic, V., Lees, E., and Reed, S. I. (1992). Association of human cyclin E with a periodic G1-S phase protein kinase. Science 257, 1958-1961.
Ellis, B. L., Hirsch, M. L., Barker, J. C., Connelly, J. P., Steininger, R. J., 3rd, and Porteus, M. H. (2013). A survey of ex vivo/in vitro transduction efficiency of mammalian primary cells and cell lines with Nine natural adeno-associated virus (AAV1-9) and one engineered adeno-associated virus serotype. Virol J 10, 74.
Fabbro, D., Cowan-Jacob, S. W., and Moebitz, H. (2015). Ten things you should know about protein kinases: IUPHAR Review 14. Br J Pharmacol 172, 2675-2700.
Fire, A., Xu, S., Montgomery, M. K., Kostas, S. A., Driver, S. E., and Mello, C. C. (1998). Potent and specific genetic interference by double-stranded RNA in Caenorhabditis elegans. Nature 391, 806-811.
Fleuren, E. D., Zhang, L., Wu, J., and Daly, R. J. (2016). The kinome ‘at large’ in cancer. Nat Rev Cancer 16, 83-98.
Futreal, P. A., Coin, L., Marshall, M., Down, T., Hubbard, T., Wooster, R., Rahman, N., and Stratton, M. R. (2004). A census of human cancer genes. Nat Rev Cancer 4, 177-183.
Gross, H. J., Verwer, B., Houck, D., Hoffman, R. A., and Recktenwald, D. (1995). Model study detecting breast cancer cells in peripheral blood mononuclear cells at frequencies as low as 10(-7). Proc Natl Acad Sci USA 92, 537-541.
Gross, H. J., Verwer, B., Houck, D., and Recktenwald, D. (1993). Detection of rare cells at a frequency of one per million by flow cytometry. Cytometry 14, 519-526.
Gross, S., Rahal, R., Stransky, N., Lengauer, C., and Hoeflich, K. P. (2015). Targeting cancer with kinase inhibitors. J Clin Invest 125, 1780-1789.
Gruber, D. F., Pieribone, V. A., Porton, B., and Kao, H. T. (2008). Strict regulation of gene expression from a high-copy plasmid utilizing a dual vector system. Protein Expr Purif 60, 53-57.
Halbert, C. L., Allen, J. M., and Miller, A. D. (2001). Adeno-associated virus type 6 (AAV6) vectors mediate efficient transduction of airway epithelial cells in mouse lungs compared to that of AAV2 vectors. J Virol 75, 6615-6624.
Koff, A., Giordano, A., Desai, D., Yamashita, K., Harper, J. W., Elledge, S., Nishimoto, T., Morgan, D. O., Franza, B. R., and Roberts, J. M. (1992). Formation and activation of a cyclin E-cdk2 complex during the G1 phase of the human cell cycle. Science 257, 1689-1694.
Moarefi, I., LaFevre-Bernt, M., Sicheri, F., Huse, M., Lee, C. H., Kuriyan, J., and Miller, W. T. (1997). Activation of the Src-family tyrosine kinase Hck by SH3 domain displacement. Nature 385, 650-653.
Obata, T., Yaffe, M. B., Leparc, G. G., Piro, E. T., Maegawa, H., Kashiwagi, A., Kikkawa, R., and Cantley, L. C. (2000). Peptide and protein library screening defines optimal substrate motifs for AKT/PKB. J Biol Chem 275, 36108-36115.
Ormo, M., Cubitt, A. B., Kallio, K., Gross, L. A., Tsien, R. Y., and Remington, S. J. (1996). Crystal structure of the Aequorea victoria green fluorescent protein. Science 273, 1392-1395.
Pagano, M., Pepperkok, R., Lukas, J., Baldin, V., Ansorge, W., Bartek, J., and Draetta, G. (1993). Regulation of the cell cycle by the cdk2 protein kinase in cultured human fibroblasts. J Cell Biol 121, 101-111.
Patel, A., Sabbineni, H., Clarke, A., and Somanath, P. R. (2016). Novel roles of Src in cancer cell epithelial-to-mesenchymal transition, vascular permeability, microinvasion and metastasis. Life Sci 157, 52-61.
Pavoor, T. V., Cho, Y. K., and Shusta, E. V. (2009). Development of GFP-based biosensors possessing the binding properties of antibodies. Proc Natl Acad Sci USA 106, 11895-11900.
Pedrali-Noy, G., Spadari, S., Miller-Faures, A., Miller, A. O., Kruppa, J., and Koch, G. (1980). Synchronization of HeLa cell cultures by inhibition of DNA polymerase alpha with aphidicolin. Nucleic Acids Res 8, 377-387.
Peelle, B., Lorens, J., Li, W., Bogenberger, J., Payan, D. G., and Anderson, D. C. (2001). Intracellular protein scaffold-mediated display of random peptide libraries for phenotypic screens in mammalian cells. Chem Biol 8, 521-534.
Ray, M., Tang, R., Jiang, Z., and Rotello, V. M. (2015). Quantitative tracking of protein trafficking to the nucleus using cytosolic protein delivery by nanoparticle-stabilized nanocapsules. Bioconjug Chem 26, 1004-1007.
Redmond, T., Brott, B. K., Jove, R., and Welsh, M. J. (1992). Localization of the viral and cellular Src kinases to perinuclear vesicles in fibroblasts. Cell Growth Differ 3, 567-576.
Rickles, R. J., Botfield, M. C., Weng, Z., Taylor, J. A., Green, O. M., Brugge, J. S., and Zoller, M. J. (1994). Identification of Src, Fyn, Lyn, PI3K and Abl SH3 domain ligands using phage display libraries. Embo J 13, 5598-5604.
Rickles, R. J., Botfield, M. C., Zhou, X. M., Henry, P. A., Brugge, J. S., and Zoller, M. J. (1995). Phage display selection of ligand residues important for Src homology 3 domain binding specificity. Proc Natl Acad Sci USA 92, 10909-10913.
Saito, S., Frank, G. D., Mifune, M., Ohba, M., Utsunomiya, H., Motley, E. D., Inagami, T., and Eguchi, S. (2002). Ligand-independent trans-activation of the platelet-derived growth factor receptor by reactive oxygen species requires protein kinase C-delta and c-Src. J Biol Chem 277, 44695-44700.
Sayroo, R., Nolasco, D., Yin, Z., Colon-Cortes, Y., Pandya, M., Ling, C., and Aslanidi, G. (2016). Development of novel AAV serotype 6 based vectors with selective tropism for human cancer cells. Gene Ther 23, 18-25.
Scott, M. P., and Miller, W. T. (2000). A peptide model system for processive phosphorylation by Src family kinases. Biochemistry 39, 14531-14537.
Shaffer, A. T. (2014). Many Companies Join Race to Create Novel CTC Technology. In OncLive (Oncology Live [OncLive.com]).
Sibilia, M., Kroismayr, R., Lichtenberger, B. M., Natarajan, A., Hecking, M., and Holcmann, M. (2007). The epidermal growth factor receptor: from development to tumorigenesis. Differentiation 75, 770-787.
Songyang, Z., Carraway, K. L., 3rd, Eck, M. J., Harrison, S. C., Feldman, R. A., Mohammadi, M., Schlessinger, J., Hubbard, S. R., Smith, D. P., Eng, C., and et al. (1995). Catalytic specificity of protein-tyrosine kinases is critical for selective signalling. Nature 373, 536-539.
Sun, X., Majumder, P., Shioya, H., Wu, F., Kumar, S., Weichselbaum, R., Kharbanda, S., and Kufe, D. (2000). Activation of the cytoplasmic c-Abl tyrosine kinase by reactive oxygen species. J Biol Chem 275, 17237-17240.
Takeda, H., Kawamura, Y., Miura, A., Mori, M., Wakamatsu, A., Yamamoto, J., Isogai, T., Matsumoto, M., Nakayama, K. I., Natsume, T., Nomura, N., and Goshima, N. (2010). Comparative analysis of human SRC-family kinase substrate specificity in vitro. J Proteome Res 9, 5982-5993.
Thakali, K., Davenport, L., Fink, G. D., and Watts, S. W. (2007). Cyclooxygenase, p38 mitogen-activated protein kinase (MAPK), extracellular signal-regulated kinase MAPK, Rho kinase, and Src mediate hydrogen peroxide-induced contraction of rat thoracic aorta and vena cava. J Pharmacol Exp Ther 320, 236-243.
Till, J. H., Chan, P. M., and Miller, W. T. (1999). Engineering the substrate specificity of the Abl tyrosine kinase. J Biol Chem 274, 4995-5003.
U.S.-Cancer-Statistics-Working-Group (2017). United States Cancer Statistics: 1999-2014 Incidence and Mortality Web-based Report. Atlanta: U.S. Department of Health and Human Services, Centers for Disease Control and Prevention and National Cancer Institute; 2017. Available at: www.cdc.gov/uscs.
Uhler, M. D., Carmichael, D. F., Lee, D. C., Chrivia, J. C., Krebs, E. G., and McKnight, G. S. (1986). Isolation of cDNA clones coding for the catalytic subunit of mouse cAMP-dependent protein kinase. Proc Natl Acad Sci USA 83, 1300-1304.
Zhang, Z., Ramnath, N., and Nagrath, S. (2015). Current Status of CTCs as Liquid Biopsy in Lung Cancer and Future Directions. Front Oncol 5, 209.

1. An isolated fluorescent polypeptide (PhosFluor) comprising an amino acid sequence at least 90% identical to SEQ ID NO: 1 (MKIKLRMEGDVNGHPFVITGEGSGKP YEGTQTVDLKVKEGGPLPFAYDILTVAFQYGNRAFTKYPADIPDYFKQSFPDGYCWERSM VFEDQGSCVVKS VISLDKKEPDCFIYDIRFKGKNFPATGPIMQKETVKWDASTQRMYERDG VLVGDAKMKLKLKGGGHYRVDIKSTYRAKGVVQYMPGNHYVDHHIEILHHDKDYNSVT VYESAEARHCRPSSKAE), and variants thereof.

2. (canceled)

3. (canceled)

4. (canceled)

5. The isolated fluorescent protein of claim 1, wherein the SEQ ID NO:1 comprises one or more mutations.

6. The isolated fluorescent protein of claim 5, wherein the mutation comprises the addition of a substrate recognition site.

7. The isolated fluorescent protein of claim 1, wherein the polypeptide further comprises at least one kinase recognition sequence.

8. (canceled)

9. (canceled)

10. The isolated fluorescent protein of claim 7, wherein the kinase recognition sequence recognizes a kinase selected from the group consisting of Protein Kinase A, Protein Kinase C, Ca2+/calmodulin-dependent protein kinase I (CAMKI), Ca2+/calmodulin-dependent protein kinase II (CAMKII) and Abl.

11. The isolated fluorescent protein of claim 1, wherein the protein further comprises at least one protein kinase docking site.

12. The isolated fluorescent protein of claim 1, wherein the protein further comprises at least one targeting sequence that directs the protein to a specific cellular compartment.

13. The isolated fluorescent polypeptide of claim 1, further comprising a second fluorescent molecule.

14. (canceled)

15. An isolated fluorescent polypeptide comprising an amino acid sequence at least 90% identical to SEQ ID NO: 1 and at least one kinase recognition sequence.

16. The isolated fluorescent polypeptide of claim 15, wherein the kinase recognition sequence is fused to the N-terminus of SEQ ID NO:1.

17. The isolated fluorescent polypeptide of claim 15, wherein the kinase recognition sequences recognizes protein kinase A (PKA).

18. An isolated fluorescent polypeptide comprising an amino acid sequence at least 90% identical to SEQ ID NO: 1 and at least one protein kinase docking site.

19. An isolated fluorescent polypeptide comprising an amino acid sequence at least 90% identical to SEQ ID NO: 1 and at least one targeting sequence that directs the protein to a specific cellular compartment.

20. A nucleic acid encoding the isolated fluorescent polypeptide of claim 1.

21. A cell comprising the nucleic acid of claim 20.

22. A method for determining whether a sample contains an activity, comprising:
obtaining a sample;
contacting the sample with the fluorescent polypeptide of claim 1; and
measuring the amount of fluorescence from said fluorescent polypeptide.

23. The method of claim 22, wherein the activity is kinase activity or phosphatase activity.

24. (canceled)

25. The method of claim 22, further comprising the step of comparing the amount of fluorescence measured from said fluorescent polypeptide with the amount of fluorescence from a control sample.

26. (canceled)

27. The method of claim 22, wherein the sample is from a subject suffering from a disease or disorder.

28. (canceled)

29. (canceled)

30. (canceled)

31. A method of screening for a compound that has kinase activity, comprising:
obtaining a first sample at a first time point and a second sample at a second time point, wherein the second time point is a time after treatment with the compound;
contacting the first sample and the second sample with the fluorescent polypeptide of claim 1; and
measuring the amount of fluorescence from said fluorescent polypeptide from the first sample and the second sample,
wherein an increase is amount of fluorescence in the second sample compared to the first sample indicates a compound that has kinase activity.
